(19) United States
(12) Patent Application Publication
(10) Pub. No.: US 2025/0302421 A1
(43) Pub. Date: Oct. 2, 2025

(54) RADIOGRAPHY APPARATUS AND CONTROL METHOD OF RADIOGRAPHY APPARATUS

(71) Applicant: FUJIFILM Corporation, Tokyo (JP)

(72) Inventor: Tatsuhiko HAYASHI, Tokyo (JP)

(73) Assignee: FUJIFILM Corporation, Tokyo (JP)

(21) Appl. No.: 19/093,222

(22) Filed: Mar. 27, 2025

(30) Foreign Application Priority Data
Mar. 29, 2024 (JP) 2024-057940
Feb. 18, 2025 (JP) 2025-024440

(51) Int. Cl. A61B 6/00; A61B 6/03; A61B 6/42; G01T 1/17

(52) U.S. Cl. CPC A61B 6/54; A61B 6/4241; G01T 1/17; A61B 6/032

(57) ABSTRACT

A radiography apparatus detects radiation emitted from a radiation source and generates a radiation image on the basis of an electric signal corresponding to the number of photons of the radiation, and the radiography apparatus includes a plurality of circuit elements having a photon counting circuit that counts the photons; a temperature measurement device that measures a temperature of a region where the plurality of circuit elements are arranged; and a processor that adjusts the temperature of the plurality of circuit elements by performing drive control of the plurality of circuit elements on the basis of a measured value of the temperature measured by the temperature measurement device, in a standby period that is a period in which the photon counting circuit does not count the photons.

### CROSS-REFERENCE TO RELATED APPLICATIONS

The present application claims priority under 35 U.S.C. § 119 to Japanese Patent Application No. 2024-057940, filed on Mar. 29, 2024, and Japanese Patent Application No. 2025-024440, filed on Feb. 18, 2025. The above applications are hereby expressly incorporated by reference, in their entirety, into the present application.

### BACKGROUND

### 1. Technical Field

The technology of the present disclosure relates to a radiography apparatus and a control method of a radiography apparatus.

### 2. Description of the Related Art

In recent years, a photon counting computed tomography (PCCT) apparatus that is a radiography apparatus equipped with a photon counting detector has been known. Unlike a charge integration detector employed in a computed tomography (CT) apparatus in the related art, the photon counting detector can count photons of incident radiation. Since the PCCT apparatus can measure energy for each photon, more information can be obtained compared to the CT apparatus in the related art.

In the PCCT apparatus, incident photons are converted into charges in a semiconductor layer, and the photon counting is performed by a photon counting circuit counting the converted charges. It is known that such a photon counting detector generates heat as a result of counting photons, and a heat generation amount is changed according to a counting rate (for example, refer to JP2018-143575A).

Since the characteristics of the photon counting circuit are changed due to a temperature change, JP2018-143575A has proposed that a heat generation amount compensation circuit that controls the heat generation amount by the photon counting circuit according to the counting rate is provided to suppress the temperature change.

### SUMMARY

However, in the technology disclosed in JP2018-143575A, a standby period in which the imaging is not performed and the photon counting circuit does not count photons is not considered. In the photon counting detector, a plurality of circuit elements including a photon counting circuit are arranged, and since the counting rate during imaging is different for each photon counting circuit, the temperature at the end of imaging is different for each circuit element. For example, in a circuit element disposed in a region where the radiation absorbance of a subject is low and the transmission amount of the radiation is large, the counting rate by the photon counting circuit is increased.

Therefore, during the standby period, the temperature may vary greatly for each circuit element. In a case where imaging is performed in a state where the temperature varies greatly for each circuit element, in the technology disclosed in JP2018-143575A, since the heat generation amount is controlled according to the counting rate, it is not possible to suppress the temperature difference of each circuit element. The temperature difference of each circuit element causes a difference in dark current noise or the like, which causes deterioration such as unevenness in a tomographic image.

Therefore, the technology according to the present disclosure provides a radiography apparatus and a control method of a radiography apparatus that can suppress a temperature difference between a plurality of circuit elements including a photon counting circuit.

A radiography apparatus according to an aspect of the technology of the present disclosure is a radiography apparatus that detects radiation emitted from a radiation source and generates a radiation image on the basis of an electric signal corresponding to the number of photons of the radiation, and the radiography apparatus includes a plurality of circuit elements having a photon counting circuit that counts the photons; a temperature measurement device that measures a temperature of a region where the plurality of circuit elements are arranged; and a processor that adjusts the temperature of the plurality of circuit elements by performing drive control of the plurality of circuit elements on the basis of a measured value of the temperature measured by the temperature measurement device, in a standby period that is a period in which the photon counting circuit does not count the photons.

It is preferable that the temperature measurement device is composed of a plurality of temperature sensors.

It is preferable that each of the plurality of temperature sensors is provided inside each of the plurality of circuit elements.

It is preferable that the processor stores, as a target value, the measured value of the temperature of each of the plurality of circuit elements measured by the temperature measurement device at a time of calibration, and performs the drive control such that the measured value approaches the target value in the standby period.

It is preferable that the processor predicts a temperature change of the plurality of circuit elements in the standby period, and performs the drive control on the basis of the predicted temperature change.

It is preferable that the processor acquires imaging plan information, and specifies, as the standby period, a period from an end of one imaging to a start of next imaging on the basis of the acquired imaging plan information.

It is preferable that the processor performs the drive control such that the temperature of each of the plurality of circuit elements becomes a target value at an end of the standby period.

It is preferable that the processor obtains an estimated temperature estimated to be reached at the end of the standby period in a case where the drive control is not performed for each of the plurality of circuit elements, and performs, in a case where any of a plurality of estimated temperatures is higher than the target value, the drive control using a highest temperature among the plurality of estimated temperatures as the target value.

It is preferable to further include at least one cooling fan for cooling the plurality of circuit elements.

It is preferable that in a case where any of a plurality of the measured values measured by the temperature measurement device is higher than a target value, the processor drives the at least one cooling fan.

It is preferable that the processor controls rotation of the at least one cooling fan such that the temperature of the plurality of circuit elements approaches the target value at an end of the standby period.

A control method of a radiography apparatus according to another aspect of the technology of the present disclosure is a control method of a radiography apparatus that detects radiation emitted from a radiation source, generates a radiation image on the basis of an electric signal corresponding to the number of photons of the radiation, and includes a plurality of circuit elements having a photon counting circuit that counts the photons, a temperature measurement device that measures a temperature of a region where the plurality of circuit elements are arranged, and a processor, the control method causing the processor to execute processing of adjusting the temperature of the plurality of circuit elements by performing drive control of the plurality of circuit elements on the basis of a measured value of the temperature measured by the temperature measurement device, in a standby period that is a period in which the photon counting circuit does not count the photons.

According to the technology of the present disclosure, it is possible to provide a radiography apparatus and a control method of a radiography apparatus that can suppress a temperature difference between a plurality of circuit elements including a photon counting circuit.

### BRIEF DESCRIPTION OF THE DRAWINGS

Exemplary embodiments according to the technique of the present disclosure will be described in detail based on the following figures, wherein:

FIG. 1 is a diagram schematically illustrating a configuration of a radiography apparatus according to a first embodiment,

FIG. 2 is a perspective view schematically illustrating a configuration of an X-ray detector,

FIG. 3 is a diagram schematically illustrating a configuration example of a detector module,

FIG. 4 is a flowchart illustrating a flow of drive control by a controller according to the first embodiment,

FIG. 5 is a diagram illustrating a relationship between a measured value of a temperature and a driving force,

FIG. 6 is a flowchart illustrating an example of a flow of calibration,

FIG. 7 is a flowchart illustrating a flow of drive control by a controller according to a second modification example,

FIG. 8 is a diagram schematically illustrating an example of drive control according to the second modification example with respect to one ASIC,

FIG. 9 is a diagram illustrating a problem of the second modification example,

FIG. 10 is a flowchart illustrating a flow of drive control by a controller according to a third modification example,

FIG. 11 is a diagram schematically illustrating a configuration of a radiography apparatus according to a second embodiment,

FIG. 12 is a flowchart illustrating a flow of drive control by a controller according to the second embodiment,

FIG. 13 is a diagram illustrating another arrangement example of a plurality of temperature sensors, and

FIG. 14 is a diagram illustrating the configuration of the photon counting circuit.

### DETAILED DESCRIPTION

Hereinafter, embodiments according to the technology of the present disclosure will be described with reference to the drawings. A radiography apparatus of the present disclosure is applied to a PCCT apparatus that detects radiation emitted from a radiation source and generates a radiation image on the basis of an electric signal corresponding to the number of photons of the radiation. In the present embodiment, a case where the radiation is X-rays will be described as an example.

### First Embodiment

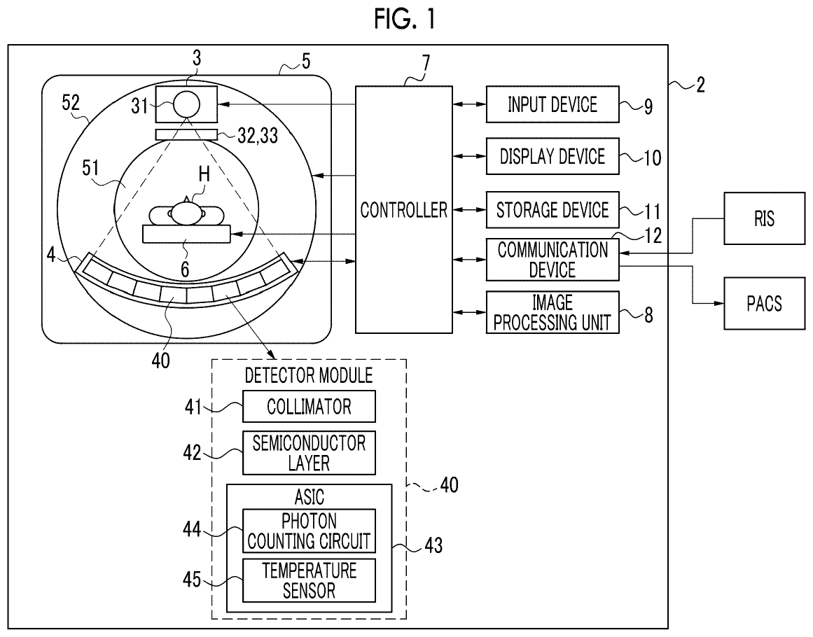

FIG. 1 schematically illustrates a configuration of a radiography apparatus 2 according to a first embodiment. The radiography apparatus 2 includes an X-ray source 3, an X-ray detector 4, a gantry 5, an examination table 6, a controller 7, and an image processing unit 8. A circular opening portion 51 for disposing the examination table 6 on which a subject H is placed is provided at the center of the gantry 5. In addition, the gantry 5 is provided with a rotation plate 52 in which the X-ray source 3 and the X-ray detector 4 are fixed at positions to face each other, and a drive mechanism (not illustrated) for rotating the rotation plate 52.

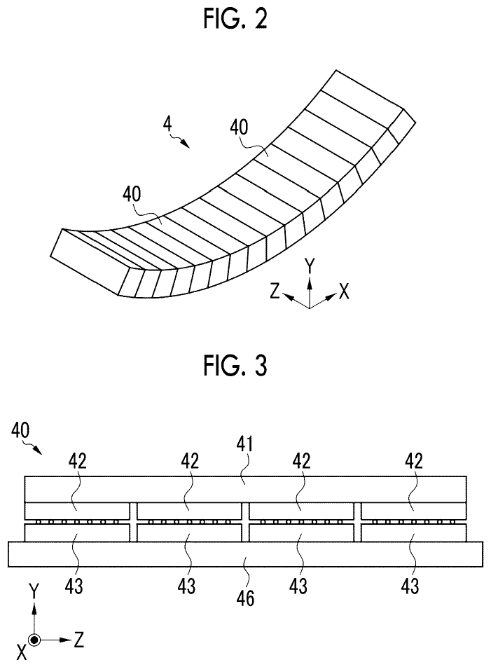

Hereinafter, in the present disclosure, a circumferential direction of the opening portion 51 is referred to as an X direction, a radial direction is referred to as a Y direction, and a central axis direction is referred to as a Z direction (refer to FIG. 2). The Z direction is orthogonal to the X direction and the Y direction, and is generally a body axis direction of the subject H.

The X-ray source 3 includes an X-ray tube 31, an X-ray filter 32, and a bowtie filter 33. The X-ray tube 31 generates X-rays, and irradiates the subject H with the generated X-rays. The X-ray filter 32 adjusts the dose of the X-rays emitted from the X-ray tube 31. The bowtie filter 33 optimizes an exposure dose by increasing the dose near the center and reducing the dose around the periphery in order to minimize the exposure dose in a peripheral portion.

As illustrated in FIG. 2, the X-ray detector 4 is configured by arranging a plurality of detector modules 40 in an arc shape in the X direction. Each of the detector modules 40 includes a collimator 41, a semiconductor layer 42, and an application specific integrated circuit (ASIC) 43.

The collimator 41 is disposed on an X-ray incident side of the semiconductor layer 42, and removes scattered rays by restricting an incident direction of the X-rays onto the semiconductor layer 42. The semiconductor layer 42 is formed of cadmium zinc telluride (CZT), cadmium telluride (CdTe), or the like, and converts the X-rays that have passed through the subject H and are incident on the semiconductor layer 42, into charges corresponding to photons and outputs the charges.

The ASIC 43 is disposed on a side of the semiconductor layer 42 opposite to the collimator 41. The ASIC 43 is a circuit element having a photon counting circuit 44. The photon counting circuit 44 counts the charges output from the semiconductor layer 42 as the number of photons, and outputs a counting signal. Note that electrodes for applying a high voltage to the semiconductor layer 42 are formed on an upper surface and a lower surface of the semiconductor layer 42. The semiconductor layer 42 is provided with a plurality of pixels by patterning the electrodes on the lower surface side of the semiconductor layer 42. The photon counting circuit 44 counts photons for each pixel, and outputs the counting signal. The counting signal corresponds to an “electric signal corresponding to the number of photons” according to the technology of the present disclosure.

In addition, a temperature sensor 45 that measures a temperature of the ASIC 43 and outputs a measured value is provided inside the ASIC 43. In the ASIC 43, the temperature is changed with a temperature change of the semiconductor layer 42 caused by the flow of the current in a case where photons are incident on the semiconductor layer 42. The temperature change of the ASIC 43 at the time of the X-rays incidence depends on the counting rate of the photons by the photon counting circuit 44. A plurality of temperature sensors 45 provided in the radiography apparatus 2 are an example of a “temperature measurement device” according to the technology of the present disclosure.

The controller 7 is composed of a processor such as a central processing unit (CPU). The controller 7 controls the operations of the X-ray source 3, the X-ray detector 4, the gantry 5, and the examination table 6. Specifically, the controller 7 controls the irradiation of the X-rays from the X-ray tube 31 of the X-ray source 3, the detection of the X-rays by the X-ray detector 4, the rotation of the rotation plate 52 of the gantry 5, and the movement of the examination table 6. In addition, the controller 7 acquires the counting signal output from the photon counting circuit 44 of the ASIC 43, and the measured value of the temperature output from the temperature sensor 45.

The image processing unit 8 is an image processing processor that generates a tomographic image (referred to as a CT image) by performing reconstruction processing on the basis of the counting signals acquired from each ASIC 43 by the controller 7. The image processing unit 8 may be configured as a part of the controller 7. The tomographic image is an example of a “radiation image” according to the technology of the present disclosure.

In addition, an input device 9, a display device 10, a storage device 11, and a communication device 12 are connected to the controller 7. The input device 9 is a device for an operator to input an operation instruction, and is composed of a keyboard, a mouse, and the like. The display device 10 is a display such as a liquid crystal display, and displays an operation screen, a tomographic image, and the like. The storage device 11 is a memory, a storage device, or the like, and stores a tomographic image, a program, various kinds of information, and the like.

The communication device 12 is a communication interface for communication with a radiology information system (RIS), picture archiving and communication systems (PACS), and the like. The communication device 12 performs transmission control in accordance with a communication protocol defined by various wired or wireless communication standards.

In addition, the ASIC 43 is configured such that the temperature is changeable by the drive control from the controller 7 in a standby period in which the photon counting circuit 44 does not count the photons. For example, the controller 7 increases the temperature of the ASIC 43 by driving the photon counting circuit 44 in an idle state to increase the power consumption. Specifically, a pseudo-pulse generation circuit is provided in the ASIC 43 to generate a pseudo-pulse for performing pseudo counting in the photon counting circuit 44, and the controller 7 drives the pseudo-pulse generation circuit to generate heat in the ASIC 43. The controller 7 can control the heat generation amount by controlling a generation rate of the pulse generated by the pseudo-pulse generation circuit. The generation rate of the pulse refers to the number of pulses generated per unit time.

In addition, instead of the pseudo-pulse generation circuit, a heat generation circuit that generates heat by itself may be provided in the ASIC 43. For example, the heat generation circuit is a circuit including a resistor. The controller 7 increases the temperature of the ASIC 43 by performing the drive control for the heat generation circuit. The controller 7 can control the heat generation amount by controlling the resistance of the heat generation circuit, the current flowing through the heat generation circuit, the ON/OFF time of the resistance, and the like.

A configuration and a control method for changing the temperature of the ASIC 43 as described above are known from JP2018-143575A.

FIG. 3 schematically illustrates a configuration example of the detector module 40. For example, the detector module 40 is a module in which four ASICs 43 are mounted on a holding substrate 46. The four ASICs 43 are arranged in the Z direction. The semiconductor layer 42 is connected to each ASIC 43. The collimator 41 is disposed on the four semiconductor layers 42. Note that the number of the ASICs 43 included in the detector module 40 are not limited to four and may be an appropriate number.

Since the counting rate of the photon during imaging is different for each photon counting circuit 44 in the ASIC 43, the temperature at the end of imaging is different for each ASIC 43. For example, in the ASIC 43 disposed in a region where the X-ray absorbance of the subject H is low and the transmission amount of the X-rays is large, the counting rate by the photon counting circuit 44 is increased. Therefore, during the standby period of the radiography apparatus 2, a temperature difference occurs between the plurality of ASICs 43. In the standby period, the counting of the photons is not performed, the temperature of each ASIC 43 is decreased, but the temperature difference of each ASIC 43 is not eliminated. Such a temperature difference deteriorates the tomographic image.

The controller 7 adjusts the temperature of each ASIC 43 by performing the drive control for each ASIC 43 in order to suppress the temperature difference of each ASIC 43 during the standby period. The standby period is a period during which the radiography apparatus 2 does not perform imaging, and is a period in which the photon counting circuit 44 does not count the photons as described above. For example, the standby period is a period from the end of one imaging to the start of the next imaging.

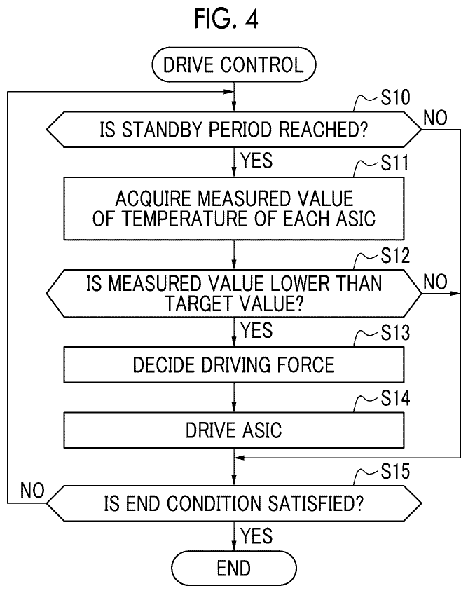

FIG. 4 illustrates a flow of the drive control by the controller 7 according to the first embodiment. In the present embodiment, the controller 7 performs the drive control for each ASIC 43 such that the temperature of each ASIC 43 becomes a predetermined target value Ta during the standby period.

First, the controller 7 determines whether or not a standby period is reached (step S10). In a case where the standby period is not reached (NO in step S10), the controller 7 causes the processing to proceed to step S15. On the other hand, in a case where the standby period is present (step S10: YES), the controller 7 acquires a measured value T of the temperature of each ASIC 43 from each temperature sensor 45 (step S11).

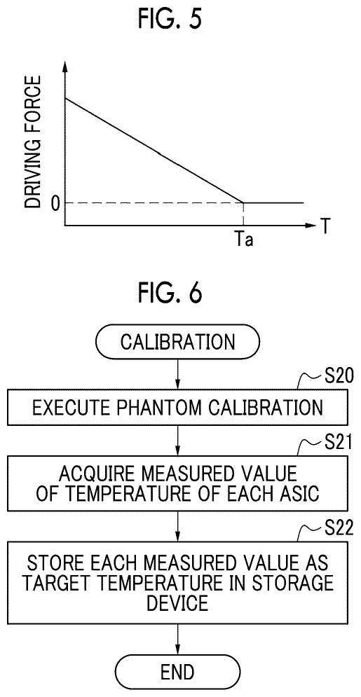

The controller 7 determines whether or not each measured value T is lower than the target value Ta (step S12). In a case where all the measured values T are higher than the target value Ta (NO in step S12), the controller 7 causes the processing to proceed to step S15. On the other hand, in a case where any measured value T is lower than the target value Ta (YES in step S12), the controller 7 decides the driving force for driving the ASIC 43 on the basis of the temperature difference between the measured value T and the target value Ta (step S13). As illustrated in FIG. 5, the controller 7 decides the larger driving force as the measured value T is lower than the target value Ta. Here, the driving force is a parameter depending on the heat generation amount, such as the generation rate of the pulse by the pseudo-pulse generation circuit, the resistance of the heat generation circuit, the current flowing through the heat generation circuit, and the ON/OFF time of the resistance. The larger the driving force, the larger the heat generation amount.

Then, the controller 7 drives the ASIC 43 with the decided driving force (step S14). The controller 7 executes steps S13 and S14 for each ASIC 43 for the ASIC 43 of which the measured value T is lower than the target value Ta.

Thereafter, the controller 7 determines whether or not an end condition is satisfied (step S15). For example, the end condition is that an end instruction input by an operator using the input device 9 is received. In a case where the end condition is not satisfied (step S15: NO), the controller 7 causes the processing to return to step S10. On the other hand, in a case where the end condition is satisfied (YES in step S15), the controller 7 ends the drive control.

As described above, the temperature of each ASIC 43 approaches the target value Ta by repeatedly executing steps S11 to S14 during the standby period. As described above, according to the present embodiment, the temperature difference between the plurality of ASICs 43 can be suppressed by the drive control during the standby period. As a result, since the temperature difference is suppressed in a case where the standby period ends and the imaging is started, the deterioration of the tomographic image caused by the difference in the dark current noise or the like is suppressed.

Hereinafter, various modification examples of the first embodiment will be described.

### First Modification Example

In the above-described embodiment, the controller 7 performs the drive control such that the temperatures of the plurality of ASICs 43 become one target value Ta, but the target value Ta may be different for each ASIC 43. For example, the controller 7 may respectively store the measured values T of the temperature measured by the respective temperature sensors 45 during calibration as the target values Ta, and may perform the drive control such that the measured value T of the temperature of each ASIC 43 approaches the target value Ta during the standby period.

For example, as illustrated in FIG. 6, the controller 7 executes phantom calibration as the calibration (step S20). The phantom calibration is processing of generating correction data by performing imaging using a phantom in which the density and the transmission length are known in order to configure the density and the transmission length of the subject H obtained by the imaging. The phantom calibration is executed at the time of shipment of the radiography apparatus 2, at the time of regular inspection, and the like.

The controller 7 acquires the measured value T of the temperature measured by each temperature sensor 45 at the time of the phantom calibration (step S21). Then, the controller 7 stores each acquired measured value T as the target value Ta in the storage device 11 (step S22).

In the standby period, the controller 7 acquires each target value Ta from the storage device 11, and performs the above-described drive control. Accordingly, at the start of imaging after the end of the standby period, each ASIC 43 can be made to approach the temperature at the time of calibration. Note that the target value Ta is not limited to the temperature used during phantom calibration; for example, it may be the temperature measured during the air calibration performed by the user each morning.

### Second Modification Example

In the above-described embodiment, the controller 7 performs the drive control such that the temperature of each ASIC 43 approaches the target value Ta and the temperature in the vicinity of the target value Ta is maintained during the standby period, but the temperature change of each ASIC 43 in the standby period may be predicted, and the drive control may be performed on the basis of the predicted temperature change. In addition, the controller 7 may specify the standby period on the basis of imaging plan information acquired from the RIS or the like via the communication device 12. For example, the imaging plan information includes an imaging timing and an imaging time in the continuous imaging.

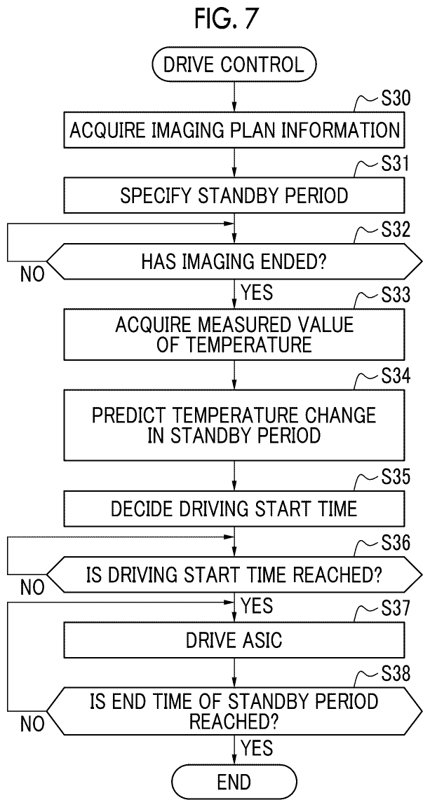

FIG. 7 illustrates a flow of the drive control by the controller 7 according to the second modification example. First, the controller 7 acquires the imaging plan information from the RIS or the like in advance (step S30), and specifies the standby period on the basis of the acquired imaging plan information (step S31).

Next, the controller 7 determines whether or not the imaging currently being performed has ended (step S32), and in a case where the imaging has not ended (step S32: NO), the determination is repeated. In a case where the imaging has ended (step S32: YES), the controller 7 acquires the measured value T of the temperature of each ASIC 43 at the end of the imaging from the temperature sensor 45 (step S33).

Next, the controller 7 predicts the temperature change of each ASIC 43 in the standby period on the basis of each measured value T (step S34). Then, the controller 7 decides a driving start time at which driving of each ASIC 43 is started, on the basis of the predicted temperature change (step S35). Thereafter, the controller 7 determines whether or not the driving start time is reached for each ASIC 43 (step S36), and in a case where the driving start time is not reached (step S36: NO), the determination is repeated.

In a case where the driving start time is reached (step S36: YES), the controller 7 drives the ASIC 43 (step S37). Then, the controller 7 determines whether or not the end time of the standby period is reached (step S38). In a case where the end time is not reached (step S38: NO), the controller 7 returns the processing to step S37 and continues the driving. In a case where the end time is reached (step S38: YES), the controller 7 ends the driving.

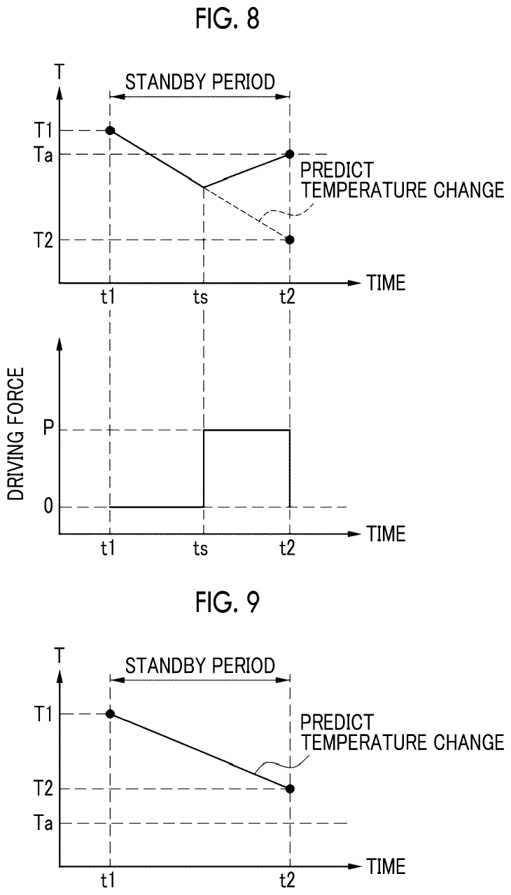

FIG. 8 schematically illustrates an example of the drive control according to the second modification example with respect to one ASIC 43. In FIG. 8, t1 is an end time of one imaging, and t2 is a start time of the next imaging. That is, a period from t1 to t2 is the standby period, and t2 is also the end time of the standby period. In addition, T1 is a measured value of the temperature at the end time t1 of the imaging. T2 is a temperature estimated to be reached in a case where the ASIC 43 is not driven during the standby period.

The controller 7 decides a driving start time ts at which it is estimated that the target value Ta is reached at the end time t2 of the standby period in a case where the ASIC 43 is driven with driving force P, on the basis of the prediction of the temperature change in the standby period. In a case where the elapsed time from the end time t1 of the imaging reaches the driving start time ts, the controller 7 drives the ASIC 43 with the driving force P. By driving the ASIC 43 with a constant driving force P until the end time t2 of the standby period, the temperature of the ASIC 43 becomes the target value Ta at the end time t2.

### Third Modification Example

In the second modification example, the temperature change of each ASIC 43 in the standby period is predicted, but as illustrated in FIG. 9, there is a case where the estimated temperature T2, which is estimated to be reached at the end time t2 of the standby period in a case where the drive control is not performed, is higher than the target value Ta. For the ASIC 43 of which the estimated temperature T2 is higher than the target value Ta, the temperature at the end time t2 cannot be set as the target value Ta. Therefore, in a case where any of the plurality of estimated temperatures T2 is higher than the target value Ta, the temperature difference of each ASIC 43 is increased.

Therefore, in the present modification example, the controller 7 obtains the estimated temperature T2, which is estimated to be reached at the end of the standby period in a case where the drive control is not performed, for each ASIC 43, and in a case where any of the plurality of estimated temperatures T2 is higher than the target value Ta, the controller 7 performs the drive control by setting the highest temperature among the plurality of estimated temperatures T2 as the target value Ta for all the ASICs 43.

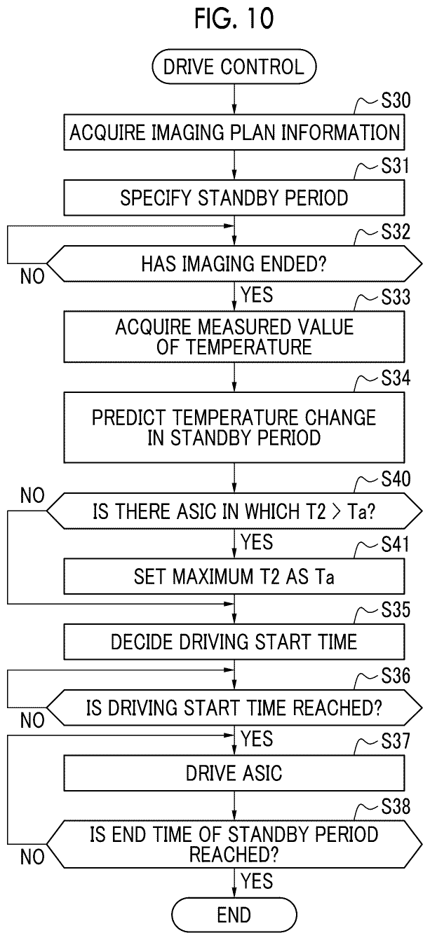

FIG. 10 illustrates a flow of the drive control by the controller 7 according to the third modification example. The drive control according to the present modification example is different from the drive control according to the second modification example in that only steps S40 and S41 are executed between steps S34 and S35.

In the present modification example, after the temperature change of each ASIC 43 in the standby period is predicted in step S34, the controller 7 determines whether or not there is an ASIC 43 in which T2>Ta (step S40). In a case where there is no ASIC 43 in which T2>Ta (step S40: NO), the controller 7 causes the processing to proceed to step S35. In a case where there is an ASIC 43 in which T2>Ta (step S40: YES), the controller 7 sets the maximum estimated temperature T2 among the estimated temperatures T2 higher than the target value Ta, as the target value Ta (step S41).

As described above, in the present modification example, even in a case where any of the plurality of estimated temperatures T2 is higher than the target value Ta, the maximum estimated temperature T2 is set as the target value Ta, and thus the temperature difference for each ASIC 43 can be suppressed.

### Second Embodiment

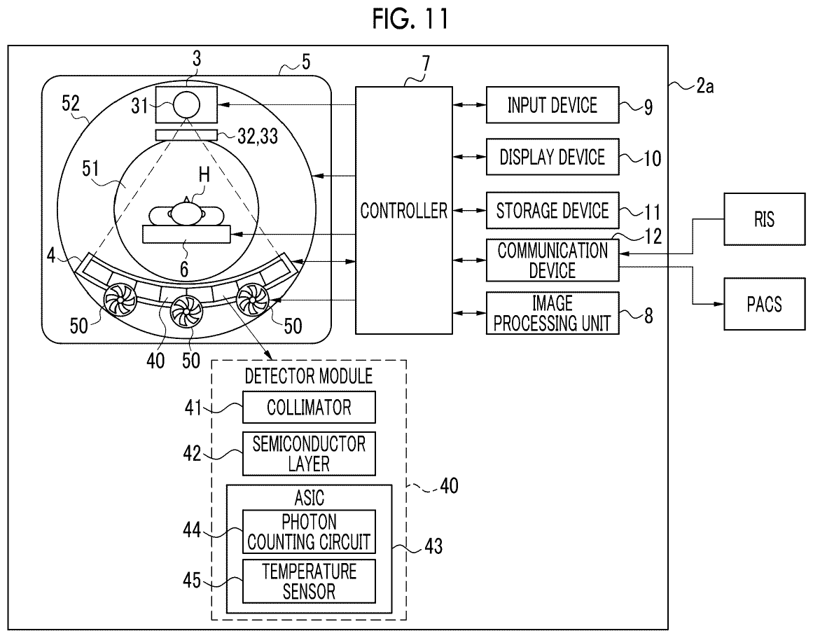

FIG. 11 schematically illustrates a configuration of a radiography apparatus 2a according to a second embodiment. In the radiography apparatus 2a, a plurality of cooling fans 50 are arranged in the vicinity of the X-ray detector 4. For example, the plurality of cooling fans 50 are arranged to blow air from the Z direction to the plurality of detector modules 40. In addition, the plurality of cooling fans 50 are driven and controlled by the controller 7. Other configurations of the radiography apparatus 2a are the same as the configurations of the radiography apparatus 2 according to the first embodiment. Note that the radiography apparatus 2a may be provided with at least one cooling fan 50.

In the present embodiment, the controller 7 drives at least one cooling fan 50 in a case where any of the measured values T of the plurality of temperatures measured for the plurality of ASICs 43 is higher than the target value Ta. In addition, the controller 7 controls the rotation of at least one cooling fan 50 such that the temperature of each ASIC 43 approaches the target value Ta at the end of the standby period.

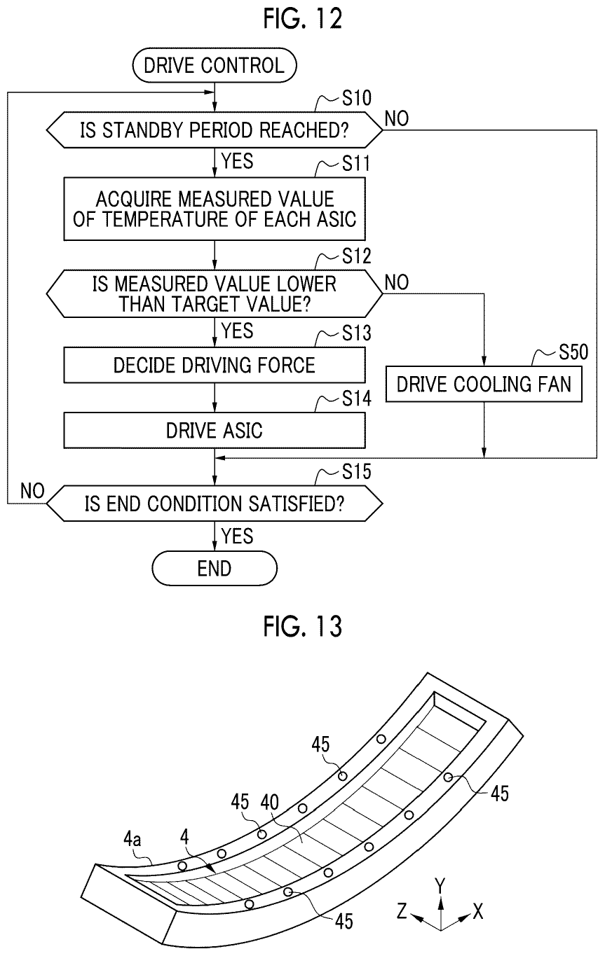

FIG. 12 illustrates a flow of the drive control by the controller 7 according to the second embodiment. The drive control according to the present embodiment is different from the drive control according to the first embodiment in that only step S50 is added. In the present embodiment, the controller 7 determines whether or not each measured value T is lower than the target value Ta in step S12, and in a case where any measured value T is higher than the target value Ta (step S12: NO), the controller 7 drives at least one cooling fan 50 (step S50). For example, all the cooling fans 50 are driven. In this case, the controller 7 controls the rotation of the cooling fan 50 according to the difference between the measured value T and the target value Ta. Specifically, the controller 7 increases the rotation speed of the cooling fan 50 as the difference between the measured value T and the target value Ta is larger. Thereafter, the controller 7 causes the processing to proceed to step S15.

As described above, in the present embodiment, in a case where the measured value T is lower than the target value Ta, the drive control of the ASIC 43 is performed, and in a case where the measured value T is higher than the target value Ta, the cooling fan 50 is driven to cool the ASIC 43. Therefore, the temperature of the ASIC 43 can be made to accurately approach the target value Ta.

Note that in a case where the second modification example of the first embodiment is applied to the second embodiment and any of the plurality of estimated temperatures T2 is higher than the target value Ta, at least one cooling fan 50 may be driven. In addition, the first modification example of the first embodiment can also be applied to the second embodiment.

Hereinafter, modification examples common to the above-described embodiments will be described.

In each of the above-described embodiments, the temperature sensor 45 is provided inside each ASIC 43, but the temperature sensor 45 may be provided outside the ASIC 43. For example, as illustrated in FIG. 13, a plurality of temperature sensors 45 may be arranged in a container 4a that accommodates the X-ray detector 4. It is preferable that the plurality of temperature sensors 45 are evenly arranged in the X direction and the Z direction. In this case, the controller 7 may acquire the measured value T of the temperature of each ASIC 43 from the temperature sensor 45 arranged in the vicinity of each ASIC 43.

In addition, the temperature measurement device is not limited to a form of being composed of the plurality of temperature sensors 45, and may be a device that can measure the temperature of the entire X-ray detector 4, such as a thermographic camera.

In the above-described embodiments, the temperature sensor 45 measures the temperature of each ASIC 43. However, it may also measure the temperature of one or more of the plurality of elements constituting the photon counting circuit 44 included in each ASIC 43. That is, “measuring a temperature of a region where the plurality of circuit elements are arranged” in the present disclosure includes measuring the temperature of one or more of the plurality of elements that constitute the circuit elements. Specific examples are described below.

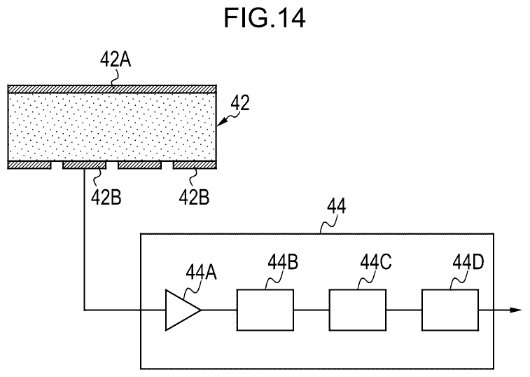

FIG. 14 illustrates the configuration of the photon counting circuit 44. The photon counting circuit 44 includes an amplification circuit 44A, a waveform shaping circuit 44B, a comparator circuit 44C, and a counter circuit 44D. These components constitute the aforementioned circuit elements.

The amplification circuit 44A amplifies the charge generated when X-ray photons enter the semiconductor layer 42. The amplification circuit 44A is connected to an upper electrode 42A formed on the semiconductor layer 42 and to one of the plurality of lower electrodes 42B. The amplification circuit 44A integrates and amplifies the charge, and outputs the resulting signal as a pulse signal.

The waveform shaping circuit 44B removes noise from the pulse signal output from the amplification circuit 44A and shapes the pulse signal into a suitable form. The comparator circuit 44C includes a plurality of comparators, each of which compares the amplitude of the pulse signal, shaped by the waveform shaping circuit 44B, with different threshold values. This allows photon energy to be classified into multiple energy bands. The counter circuit 44D includes a plurality of counters, each associated with a different energy band, for counting the number of photons in that band and outputting a corresponding counting signal.

With this configuration, the photon counting circuit 44 is capable of performing highly accurate energy discrimination and photon counting.

In the above-described embodiments, the temperature sensor 45 provided inside each ASIC 43 may measure the temperature of one of the elements among the amplification circuit 44A, the waveform shaping circuit 44B, the comparator circuit 44C, and the counter circuit 44D during the standby period. For example, the temperature sensor 45 may measure the temperature of the element that exhibits the largest temperature increase resulting from photon counting. Additionally, the temperature sensor 45 may measure the temperature of the element whose fluctuations most significantly affect the photon counting output. Furthermore, the temperature sensor 45 may measure the temperature of two or more such elements. In this case, the measured temperature may be the average or maximum value of the two or more measured temperatures.

That is, in the above-described embodiments, the temperature sensor 45 may measure the temperature of one or more elements constituting the photon counting circuit 44. In such a case, as in the first embodiment, the controller 7 may perform temperature adjustment by driving (i.e., idle-driving) one or more of the elements constituting the photon counting circuit 44 using a pseudo signal to increase the temperature. Additionally, in a manner similar to the second embodiment, the controller 7 may perform temperature adjustment by driving the cooling fan 50.

Furthermore, the photon counting circuit 44 is not limited to the above configuration and may include an A/D converter that converts analog signals such as charge signals into digital signals. In this case, the temperature sensor 45 may measure the temperature of the A/D converter. Moreover, the controller 7 may perform temperature adjustment by driving (i.e., idle-driving) the A/D converter using a pseudo signal to increase its temperature.

In the above-described embodiments, the elements constituting the photon counting circuit 44 have been described as being included within each ASIC 43, but this is not limiting. For example, the amplification circuit 44A, the waveform shaping circuit 44B, the comparator circuit 44C, the counter circuit 44D, and the A/D converter that collectively constitute the photon counting circuit 44 may be configured as discrete components in combination. In addition, some of these elements may be included within the ASIC 43, while others may be configured as external electronic components. Furthermore, the temperature sensor 45 may be used to detect the temperature of one or more of these electronic components, and the controller 7 may perform temperature adjustment by driving (i.e., idle-driving) one or more of these electronic components using a pseudo signal in order to increase their temperature. Similarly, where the photon counting circuit 44 includes an A/D converter, the A/D converter may also be configured as a discrete electronic component.

In addition, in each of the above-described embodiments, the X-rays have been described as an example of the radiation, but γ-rays may be used as the radiation.

In addition, in the above-described embodiment, various processors described below can be used as the hardware structure of the controller 7. The various processors include, in addition to a CPU that is a general-purpose processor that executes software (program) to function as various processing units, a programmable logic device (PLD) of which a circuit configuration can be changed after manufacturing, such as a field-programmable gate array (FPGA), and a dedicated electric circuit that is a processor having a circuit configuration dedicatedly designed for executing specific processing, such as an ASIC.

Various types of processing described above may be executed by one of the various processors or may be executed by a combination of two or more processors (for example, a combination of a plurality of FPGAs or a CPU and an FPGA) of the same type or different types. In addition, a plurality of processing units may be configured by one processor. As an example where a plurality of processing units are composed of one processor, there is a form in which a processor that realizes all functions of a system including a plurality of processing units into one integrated circuit (IC) chip is used, such as a system on a chip (SOC).

It is possible to understand technologies described in the following supplementary notes from the above description.

### Supplementary Note 1

A radiography apparatus that detects radiation emitted from a radiation source and generates a radiation image on the basis of an electric signal corresponding to the number of photons of the radiation, the radiography apparatus comprising:
a plurality of circuit elements having a photon counting circuit that counts the photons;
a temperature measurement device that measures a temperature of a region where the plurality of circuit elements are arranged; and
a processor that adjusts the temperature of the plurality of circuit elements by performing drive control of the plurality of circuit elements on the basis of a measured value of the temperature measured by the temperature measurement device, in a standby period that is a period in which the photon counting circuit does not count the photons.

### Supplementary Note 2

The radiography apparatus according to Supplementary Note 1,
wherein the temperature measurement device is composed of a plurality of temperature sensors.

### Supplementary Note 3

The radiography apparatus according to Supplementary Note 2,
wherein each of the plurality of temperature sensors is provided inside each of the plurality of circuit elements.

### Supplementary Note 4

The radiography apparatus according to any one of Supplementary Notes 1 to 3,
wherein the processor
   stores, as a target value, the measured value of the temperature of each of the plurality of circuit elements measured by the temperature measurement device at a time of calibration, and
   performs the drive control such that the measured value approaches the target value in the standby period.

### Supplementary Note 5

The radiography apparatus according to any one of Supplementary Notes 1 to 3,
wherein the processor
   predicts a temperature change of the plurality of circuit elements in the standby period, and
   performs the drive control on the basis of the predicted temperature change.

### Supplementary Note 6

The radiography apparatus according to Supplementary Note 5,
wherein the processor
   acquires imaging plan information, and
   specifies, as the standby period, a period from an end of one imaging to a start of next imaging on the basis of the acquired imaging plan information.

### Supplementary Note 7

The radiography apparatus according to Supplementary Note 5 or 6,
wherein the processor performs the drive control such that the temperature of each of the plurality of circuit elements becomes a target value at an end of the standby period.

### Supplementary Note 8

The radiography apparatus according to Supplementary Note 7,
wherein the processor
   obtains an estimated temperature estimated to be reached at the end of the standby period in a case where the drive control is not performed for each of the plurality of circuit elements, and
   performs, in a case where any of a plurality of estimated temperatures is higher than the target value, the drive control using a highest temperature among the plurality of estimated temperatures as the target value.

### Supplementary Note 9

The radiography apparatus according to Supplementary Note 1, further comprising:
at least one cooling fan for cooling the plurality of circuit elements.

### Supplementary Note 10

The radiography apparatus according to Supplementary Note 9,
wherein in a case where any of a plurality of the measured values measured by the temperature measurement device is higher than a target value, the processor drives the at least one cooling fan.

### Supplementary Note 11

The radiography apparatus according to Supplementary Note 10,
wherein the processor controls rotation of the at least one cooling fan such that the temperature of the plurality of circuit elements approaches the target value at an end of the standby period.

What is claimed is:

1. A radiography apparatus that detects radiation emitted from a radiation source and generates a radiation image on the basis of an electric signal corresponding to the number of photons of the radiation, the radiography apparatus comprising:
a plurality of circuit elements having a photon counting circuit that counts the photons;
a temperature measurement device that measures a temperature of a region where the plurality of circuit elements are arranged; and
a processor that adjusts the temperature of the plurality of circuit elements by performing drive control of the plurality of circuit elements on the basis of a measured value of the temperature measured by the temperature measurement device, in a standby period that is a period in which the photon counting circuit does not count the photons.

2. The radiography apparatus according to claim 1,
wherein the temperature measurement device is composed of a plurality of temperature sensors.

3. The radiography apparatus according to claim 2,
wherein each of the plurality of temperature sensors is provided inside each of the plurality of circuit elements.

4. The radiography apparatus according to claim 1,
wherein the processor
stores, as a target value, the measured value of the temperature of each of the plurality of circuit elements measured by the temperature measurement device at a time of calibration, and
performs the drive control such that the measured value approaches the target value in the standby period.

5. The radiography apparatus according to claim 1,
wherein the processor
predicts a temperature change of the plurality of circuit elements in the standby period, and
performs the drive control on the basis of the predicted temperature change.

6. The radiography apparatus according to claim 5,
wherein the processor
acquires imaging plan information, and
specifies, as the standby period, a period from an end of one imaging to a start of next imaging on the basis of the acquired imaging plan information.

7. The radiography apparatus according to claim 5,
wherein the processor performs the drive control such that the temperature of each of the plurality of circuit elements becomes a target value at an end of the standby period.

8. The radiography apparatus according to claim 7,
wherein the processor
obtains an estimated temperature estimated to be reached at the end of the standby period in a case where the drive control is not performed for each of the plurality of circuit elements, and
performs, in a case where any of a plurality of estimated temperatures is higher than the target value, the drive control using a highest temperature among the plurality of estimated temperatures as the target value.

9. The radiography apparatus according to claim 1, further comprising:
at least one cooling fan for cooling the plurality of circuit elements.

10. The radiography apparatus according to claim 9,
wherein in a case where any of a plurality of the measured values measured by the temperature measurement device is higher than a target value, the processor drives the at least one cooling fan.

11. The radiography apparatus according to claim 10,
wherein the processor controls rotation of the at least one cooling fan such that the temperature of the plurality of circuit elements approaches the target value at an end of the standby period.

12. A control method of a radiography apparatus that detects radiation emitted from a radiation source, generates a radiation image on the basis of an electric signal corresponding to the number of photons of the radiation, and includes a plurality of circuit elements having a photon counting circuit that counts the photons, a temperature measurement device that measures a temperature of a region where the plurality of circuit elements are arranged, and a processor, the control method comprising:
causing the processor to execute processing of adjusting the temperature of the plurality of circuit elements by performing drive control of the plurality of circuit elements on the basis of a measured value of the temperature measured by the temperature measurement device, in a standby period that is a period in which the photon counting circuit does not count the photons.
